(19) United States
(12) Patent Application Publication
(10) Pub. No.: US 2022/0130556 A1
(43) Pub. Date: Apr. 28, 2022

(54) HEALTH MANAGEMENT APPARATUS AND HEALTH MANAGEMENT SYSTEM

(71) Applicant: CANON MEDICAL SYSTEMS CORPORATION, Otawara-shi (JP)

(72) Inventors: Fuminori FUJITA, Nasushiobara (JP); Yuichi KAWABATA, Nasushiobara (JP); Kei MORI, Brookline, MA (US); Junko SHIBATA, Nasushiobara (JP)

(73) Assignee: CANON MEDICAL SYSTEMS CORPORATION, Otawara-shi (JP)

(21) Appl. No.: 17/511,873

(22) Filed: Oct. 27, 2021

(30) Foreign Application Priority Data
Oct. 28, 2020 (JP) 2020-180544

(51) Int. Cl. G16H 50/80; G16H 10/60; G16H 50/70; G16H 50/20; G16H 10/20; G16H 50/30; A61B 5/11; A61B 5/00

(52) U.S. Cl. CPC G16H 50/80; G16H 10/60; G16H 50/70; G16H 50/20; G16H 10/20; G16H 20/10; A61B 5/1118; A61B 5/7405; A61B 5/742; A61B 5/7275; G16H 50/30

(57) ABSTRACT

A health management apparatus according to an embodiment is provided with processing circuitry. The processing circuitry is configured to acquire vital data and activity history information of a subject. The processing circuitry is configured to estimate a disease candidate of the subject based on the acquired vital data and the acquired activity history information. The processing circuitry is configured to cause a display, a speaker, or a terminal to output a notification regarding the estimated disease candidate or an interview item for determining a disease of the subject from the disease candidate. The activity history information includes at least information on a temperature of a space around the subject, information on a movement history of the subject, and a history of position information of the subject.

### CROSS-REFERENCE TO RELATED APPLICATIONS

This application is based upon and claims the benefit of priority from Japanese Patent Application No. 2020-180544, filed on Oct. 28, 2020; the entire contents of which are incorporated herein by reference.

### FIELD

Embodiments described herein relate generally to a health management apparatus and a health management system.

### BACKGROUND

In the related art, a technique for interviewing patients online by the patients inputting responses to inquiries that are presented through networks such as the Internet is known. In such a technique, the patient's disease is generally identified by the patient oneself responding a large number of interview items.

### BRIEF DESCRIPTION OF THE DRAWINGS

FIG. 1 is a block diagram illustrating an example of a configuration of a health management system according to a first embodiment;

FIG. 2 is a diagram illustrating an example of a usage form of the health management system according to the first embodiment;

FIG. 3 is a diagram illustrating an example of disease-specific scoring according to the first embodiment;

FIG. 4 is a flowchart illustrating an example of a flow of health management processing executed by a health management apparatus according to the first embodiment;

FIG. 5 is a flowchart illustrating another example of a flow of health management processing executed by the health management apparatus according to the first embodiment;

FIG. 6 is a diagram illustrating an example of a usage form of a health management system according to a third embodiment;

FIG. 7 is a diagram illustrating an example of a usage form of a health management system according to a fourth embodiment;

FIG. 8 is a diagram illustrating an example of a method of estimating a risk of contracting a disease according to the fourth embodiment;

FIG. 9 is a diagram illustrating an example of an infection preliminary group according to a fifth embodiment; and

FIG. 10 is a diagram illustrating an example of a usage form of a health management system according to a sixth embodiment.

### DETAILED DESCRIPTION

Hereinafter, embodiments of a health management apparatus and a health management system will be described in detail with reference to the drawings.

### First Embodiment

A health management apparatus according to an embodiment includes processing circuitry. The processing circuitry acquires vital data and activity history information of a subject. The processing circuitry estimates a disease candidate of the subject based on the acquired vital data and the activity history information. The processing circuitry causes a display, a speaker, or a terminal to output a notification regarding the estimated disease candidate or an interview item for determining a disease of the subject from the disease candidate. The activity history information includes at least information on a temperature of a space around the subject, information on a movement history of the subject, and a history of position information of the subject.

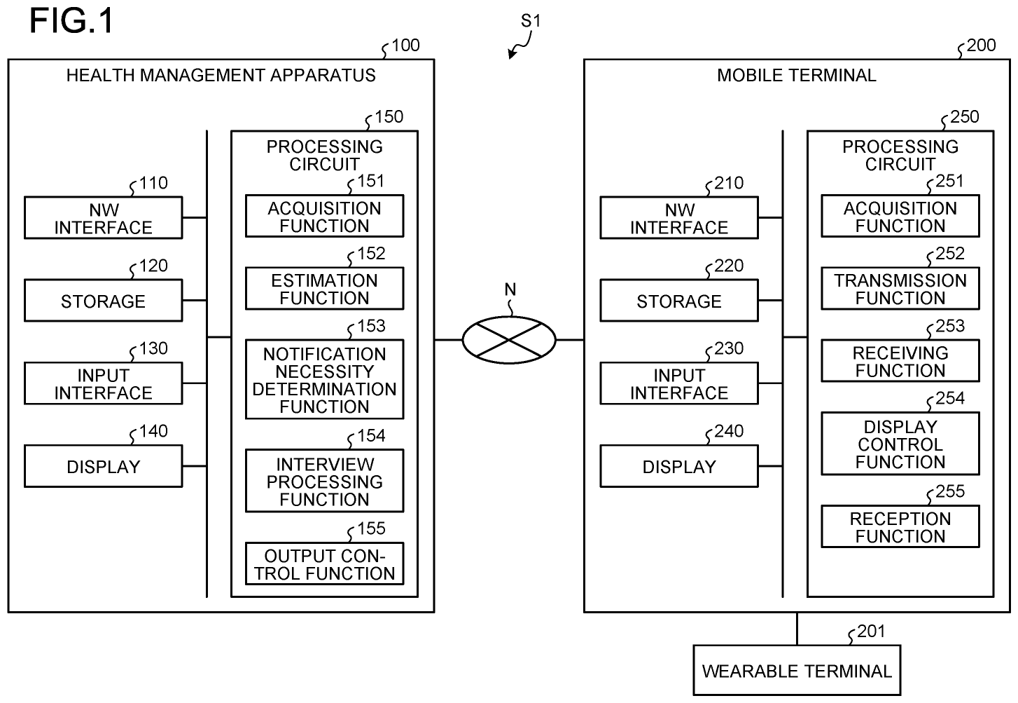

FIG. 1 is a block diagram illustrating an example of a configuration of a health management system S1 according to a first embodiment. As illustrated in FIG. 1, the health management system S1 is provided with a health management apparatus 100 and at least one mobile terminal 200. The health management apparatus 100 and the mobile terminal 200 are connected through a network N. The network N is, for example, the Internet or the like.

In addition, the mobile terminal 200 is connected to a wearable terminal 201 in a communicable manner. A connection format between the mobile terminal 200 and the wearable terminal 201 is, for example, a wireless connection using Bluetooth (registered trademark) or the like, but the connection format is not limited thereto. The wearable terminal 201 may also be included in the health management system S1. In addition, the wearable terminal 201 may be connected to the health management apparatus 100 through the network N.

In addition, the health management apparatus 100 may be connected to various information processing devices such as an electronic medical record system (not illustrated) through the network N.

The health management apparatus 100 is, for example, a server device or a computer such as a personal computer (PC).

In addition, the mobile terminal 200 is, for example, a computer such as a tablet terminal, a smartphone, or a PC. The mobile terminal 200 is an example of a terminal in the present embodiment. Alternatively, the mobile terminal 200 and the wearable terminal 201 may be collectively referred to as an example of the terminal in the present embodiment.

In addition, the wearable terminal 201 is a terminal that can be attached to the subject, and measures the vital data of the subject. The mobile terminal 200 and the wearable terminal 201 may be configured as a single device.

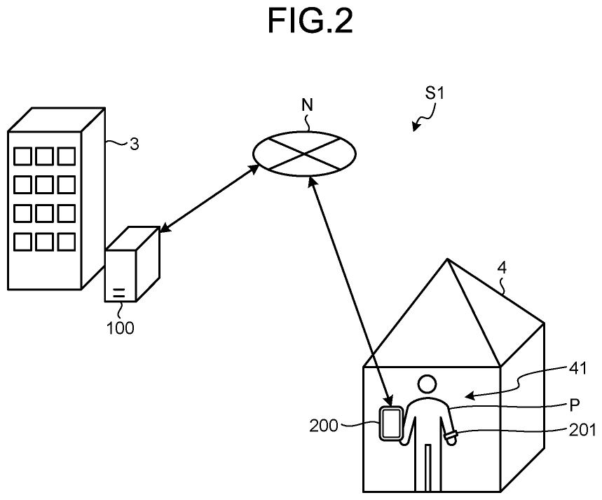

FIG. 2 is a diagram illustrating an example of a usage form of the health management system S1 according to the first embodiment. As illustrated in FIG. 2, the health management apparatus 100 is installed in, for example, a medical institution 3 such as a hospital.

A patient P is a health management subject in the health management system S1 and the health management apparatus 100 of the present embodiment. Hereinafter, the health management subject is simply referred to as a subject.

The patient P may not be present in the medical institution 3, and may be present in a room 41 of a home 4, for example. Alternatively, the patient P may be present outside.

In addition, as in an example illustrated in FIG. 2, the mobile terminal 200 is held by the patient P. In a case where the mobile terminal 200 and the wearable terminal 201 are separate devices as in the examples of FIGS. 1 and 2, the mobile terminal 200 may not be in contact with the body of the patient P at all times. The term “hold” in the present embodiment is not limited to the case where the patient P wears the mobile terminal 200 as illustrated in FIG. 2, as long as the mobile terminal 200 can be used.

The mobile terminal 200 transmits information on the patient P to the health management apparatus 100. In addition, the mobile terminal 200 supports health management of the patient P by displaying a notification regarding a disease of the patient P transmitted from the health management apparatus 100 or displaying an interview screen for identifying a disease of the patient P.

The wearable terminal 201 is a measuring instrument that can be attached to the body of the patient P and measures vital data of the patient P. In FIG. 2, the wearable terminal 201 is a wristwatch type, but the form of the wearable terminal 201 is not limited thereto. In addition, measuring instruments other than the wearable terminal 201 may be used. For example, a body temperature meter, a room temperature meter, a smart home appliance provided with various sensors, or the like, which is capable of communicating with the mobile terminal 200, may be used as the measuring instrument.

Examples of the vital data of the patient P measured by the wearable terminal 201 include body temperature, blood pressure, pulse, oxygen saturation in blood, sleep time, and the like. One wearable terminal 201 may have a plurality of functions, or a plurality of wearable terminals 201 may be used.

The wearable terminal 201 also collects information on activities of the patient P. The information on the activities of the patient P collected by the wearable terminal 201 includes, for example, the number of steps of the patient P, acceleration of the patient P, position information of the patient P, a temperature of a space around the patient P, and the like. The temperature of the space around the patient P is a temperature of a space within a defined distance from the patient P. The defined distance is not particularly limited. For example, in a case where the patient P is present in the room 41, a temperature of a space around the patient P is such that a room temperature of the room 41 is the temperature around the patient. In addition, the position information includes, for example, GPS information of the wearable terminal 201. Alternatively, the position information may be GPS information of the mobile terminal 200.

The wearable terminal 201 transmits the measured vital data and the information on the activities of the patient P to the mobile terminal 200. In addition, these vital data and information on the activities of the patient P may be measured by the mobile terminal 200.

Returning to FIG. 1, the mobile terminal 200 is provided with a network (NW) interface 210, a storage 220, an input interface 230, a display 240, and a processing circuit 250. The NW interface 210 is connected to the processing circuit 250 and controls the transmission and communication of various types of data performed between the health management apparatus 100 and the mobile terminal 200. The NW interface 210 is realized by a network card, a network adapter, a network interface controller (NIC), or the like. In addition, the NW interface 210 controls the transmission and communication of various types of data performed between the mobile terminal 200 and the wearable terminal 201.

The storage 220 is connected to the processing circuit 250 and stores various types of information and computer programs used in the processing circuit 250.

The storage 220 is realized by, for example, a random access memory (RAM), a semiconductor memory element such as a flash memory, a hard disk, an optical disk, or the like. The storage 220 is also referred to as a storage unit.

The input interface 230 is realized by a trackball, a switch button, a mouse, a keyboard, a touch pad for performing input operations by touching an operation surface, a touch screen in which a display screen and a touch pad are integrated, a non-contact input circuit using an optical sensor, a voice input circuit, or the like. The input interface 230 is connected to the processing circuit 250, converts an input operation received from a user into an electric signal, and outputs the electric signal to the processing circuit 250.

In the present specification, the input interface 230 is not limited to only those provided with physical operating parts such as a mouse and a keyboard. For example, an example of the input interface 230 includes an electric signal processing circuit that receives an electric signal corresponding to an input operation from an external input device separately provided from a device and outputs this electric signal to the processing circuit 250.

The display 240 is a liquid crystal display, an organic electro-luminescence (OEL) display, or the like. The input interface 230 and the display 240 may be integrated. For example, the input interface 230 and the display 240 may be realized by a touch panel. The display 240 is an example of a display unit or an output unit. Alternatively, the entire mobile terminal 200 may be used as an example of a display unit or an output unit.

The processing circuit 250 is processing circuitry that realizes a function corresponding to each computer program by reading out computer programs from the storage 220 and executing the computer programs. The processing circuit 250 of the present embodiment is provided with an acquisition function 251, a transmission function 252, a receiving function 253, a display control function 254, and a reception function 255. The acquisition function 251 is an example of an acquisition unit or a first acquisition unit. The transmission function 252 is an example of a transmission unit or a first transmission unit. The receiving function 253 is an example of a receiving unit. The display control function 254 is an example of a display control unit or an output control unit.

Here, for example, each processing function of the acquisition function 251, the transmission function 252, the receiving function 253, the display control function 254, and the reception function 255, which are components of the processing circuit 250, is stored in the storage 220 in the form of a computer program capable of being executed by a computer. The processing circuit 250 is processing circuitry. For example, the processing circuit 250 realizes a function corresponding to each computer program by reading out computer programs from the storage 220 and executing the computer programs. In other words, the processing circuit 250 in a state where each computer program is read out includes each function illustrated in the processing circuit 250 of FIG. 1. It is described in FIG. 1 that the processing functions performed by the acquisition function 251, the transmission function 252, the receiving function 253, the display control function 254, and the reception function 255 are realized by the single processing circuitry, but a plurality of independent processing circuitries may be combined to form the processing circuit 250, and each processing circuitry may execute a computer program to realize the functions. In addition, it is described in FIG. 1 that the single storage 220 stores the computer program corresponding to each processing function, but a plurality of storage may be distributed and disposed, and the processing circuit 250 may be configured to read out the corresponding computer program from the individual storage.

The acquisition function 251 acquires the vital data of the patient P and the information on the activities of the patient P from the wearable terminal 201.

The transmission function 252 transmits the vital data of the patient P and the information on the activities of the patient P to the health management apparatus 100 through the NW interface 210. The vital data of the patient P and the information on the activities of the patient P transmitted by the transmission function 252 are not limited to the vital data and information on the activities of the patient P acquired by the acquisition function 251, and may be the vital data and information on the activities of the patient P measured by the mobile terminal 200.

The transmission function 252 transmits a request of the patient P to start an interview, which is received by the reception function 256 described later, and contents of a response input from the interview screen by the patient P to the health management apparatus 100. In addition, the transmission function 252 transmits attribute information, information on a medical history, and a dosing history or a medication history of the patient P to the health management apparatus 100.

The information on a medical history includes, for example, information on illnesses that the patient P has contracted so far. In addition, the information on the medical history may do not necessarily include all of medical histories, and may be, for example, information indicating whether or not the patient P has a specific chronic disease.

The receiving function 253 acquires a notification regarding a disease of the patient P or information on interview items from the health management apparatus 100 through the NW interface 210.

The display control function 254 displays an interview screen on the display 240. The interview screen is a screen on which inquiries for determining a disease of the patient P are displayed and responses of the patient P to the inquiries can be input.

In addition, the display control function 254 displays a notification regarding the disease of the patient P on the display 240.

The reception function 255 receives an operation by the patient P through the input interface 230. For example, the reception function 255 receives an operation of the request to start the interview by the patient P. In addition, the reception function 255 receives an input of a response to an inquiry item of the interview by the patient P. In addition, the reception function 255 receives an input of patient's own attribute information, information on medical history, and dosing history or medication history, by the patient P.

Next, a configuration of the health management apparatus 100 will be described. The health management apparatus 100 is provided with a NW interface 110, a storage 120, an input interface 130, a display 140, and a processing circuit 150. Hardware configurations of the NW interface 110, the storage 120, the input interface 130, and the display 140 are, for example, the same as the hardware configurations of the NW interface 210, the storage 220, the input interface 230, and the display 240 of the mobile terminal 200 described above.

The NW interface 110 is connected to the processing circuit 150 and controls the transmission and communication of various types of data performed between the health management apparatus 100 and the mobile terminal 200.

The storage 120 is connected to the processing circuit 150 and stores various types of information and computer programs used in the processing circuit 150. In addition, in the present embodiment, the storage 120 stores the vital data of the patient P and the information on the activities of the patient P transmitted from the mobile terminal 200 in association with a measurement time.

The input interface 130 is connected to the processing circuit 150, converts an input operation received from a user into an electric signal, and outputs the electric signal to the processing circuit 150.

The processing circuit 150 is processing circuitry that realizes a function corresponding to each computer program by reading out a computer program from the storage 120 and executing the computer program. The processing circuit 150 of the present embodiment is provided with an acquisition function 151, an estimation function 152, a notification necessity determination function 153, an interview processing function 154, and an output control function 155. The acquisition function 151 is an example of an acquisition unit or a second acquisition unit. The estimation function 152 is an example of an estimation unit. The notification necessity determination function 153 is an example of a notification necessity determination unit. The interview processing function 154 is an example of an interview processing unit. The output control function 155 is an example of an output control unit or a second transmission unit.

Here, for example, each processing function of the acquisition function 151, the estimation function 152, the notification necessity determination function 153, the interview processing function 154, and the output control function 155, which are components of the processing circuit 150, is stored in the storage 120 in the form of a computer program capable of being executed by a computer. The processing circuit 150 is processing circuitry. For example, the processing circuit 150 realizes a function corresponding to each computer program by reading out computer programs from the storage 120 and executing the computer programs. In other words, the processing circuit 150 in a state where each computer program is read out includes each function illustrated in the processing circuit 150 of FIG. 1. It is described in FIG. 1 that the processing functions performed by the acquisition function 151, the estimation function 152, the notification necessity determination function 153, the interview processing function 154, and the output control function 155 are realized by the single processing circuitry, but a plurality of independent processing circuitries may be combined to form the processing circuit 150, and each processing circuitry may execute a computer program to realize the functions. In addition, it is described in FIG. 1 that the single storage 120 stores the computer program corresponding to each processing function, but a plurality of storage may be distributed and disposed, and the processing circuit 150 may be configured to read out the corresponding computer program from the individual storage.

In the above description, the example in which the “processing circuitry” reads out the computer program corresponding to each function from the storage and executes each function is described, but the embodiment is not limited thereto. In the present embodiment, the term “processing circuitry” refers to, for example, a circuit such as a central processing unit (CPU), a graphics processing unit (GPU), an application specific integrated circuit (ASIC), and a computer programmable logic device (for example, a simple computer programmable logic device (SPLD)), a complex computer programmable logic device (CPLD), or a field computer programmable gate array (FPGA). In a case where the processing circuitry is, for example, a CPU, the processing circuitry realizes a function by reading out and executing a computer program stored in the storage. On the other hand, in a case where the processing circuitry is an ASIC, the function is directly incorporated as a logic circuit in a circuit of the processing circuitry instead of storing the computer program in the storage 120 and 220. Each processing circuitry of the present embodiment is not limited to a case where each processing circuitry is configured as a single circuit, and a plurality of independent circuits may be combined to form one processing circuitry to realize the function. Furthermore, a plurality of components in FIG. 1 may be integrated into one processing circuitry to realize the function.

The acquisition function 151 acquires the vital data and the activity history information of the subject. In addition, the acquisition function 151 acquires the attribute information of the patient P.

The activity history information is information indicating when, where, in what environment, and what the subject did. The activity history information includes at least information on a temperature of a space around the subject, information on a movement history of the subject, and a history of position information of the subject. In the present embodiment, the activity history information further includes a dosing history or a medication history of the subject. In addition, the activity history information of the present embodiment also includes information on a medical history of the subject. Alternatively, the information on the medical history of the subject may be acquired as information different from the activity history information.

The information on the temperature of the space around the subject may be numerical data indicating a temperature, information on a change in the temperature, or the like. For example, the information on the temperature of the space around the subject may also be information indicating that the temperature of the space where the patient P is positioned has rapidly increased or decreased. The change in the temperature of the space where the patient P is positioned may be either a case where the temperature of the space changes or a case generated by the patient P moving to a space having a different temperature.

The movement history includes, for example, the number of steps, acceleration, or a moving distance. The information on the movement history is not limited to information on the movement itself performed by the patient P. For example, the information on the movement history may be information indicating whether or not the patient P has moved more intensely than usual.

The attribute information of the patient P is, for example, the age, gender, height, weight of the patient, and the like.

Furthermore, the activity history information may also include external environment information such as weather, temperature, humidity, barometric pressure in an area where the subject is positioned, amount and type of pollen in the air, amount and type of air pollutants such as PM 2.5, radiation amount, working hours of the subject, and whether or not there is a change in a living environment such as migration. In addition, the activity history information may also include meal contents of the subject, meal time, REM sleep time, non-REM sleep time, excretion timing, the number of excretion, movement speed, movement discontinuity, posture or body axis tilt, movement load, and the like. The movement discontinuity of the subject indicates, for example, an action such as dragging a leg while walking. In addition, the frequency of demonstrative pronouns extracted from a conversation record of the subject or a search history of the subject, the frequency of searching for specific words, and the like may be included in the activity history information.

In the present embodiment, the acquisition function 151 acquires these vital data and activity history information of the subject from, for example, the mobile terminal 200. An acquisition source of the vital data and activity history information of the subject is not limited to the mobile terminal 200. For example, the acquisition function 151 may acquire such information from an electronic medical record system or the like. The acquired vital data and activity history information of the subject may be vital data and activity history information automatically measured by the wearable terminal 201, or may be vital data and activity history information input to the mobile terminal 200 or the electronic medical record system by the patient P, a doctor, or the like.

The estimation function 152 estimates a disease candidate of the patient P who is a subject, based on the acquired vital data and activity history information.

For example, the estimation function 152 scores a degree of a possibility of contracting a disease for each item included in the vital data and activity history information of the patient P based on disease information in which the vital data and activity history information are associated with a disease that is likely to be contracted by the patient P, and estimates a disease candidate based on the scoring result. The disease information is stored in, for example, the storage 120.

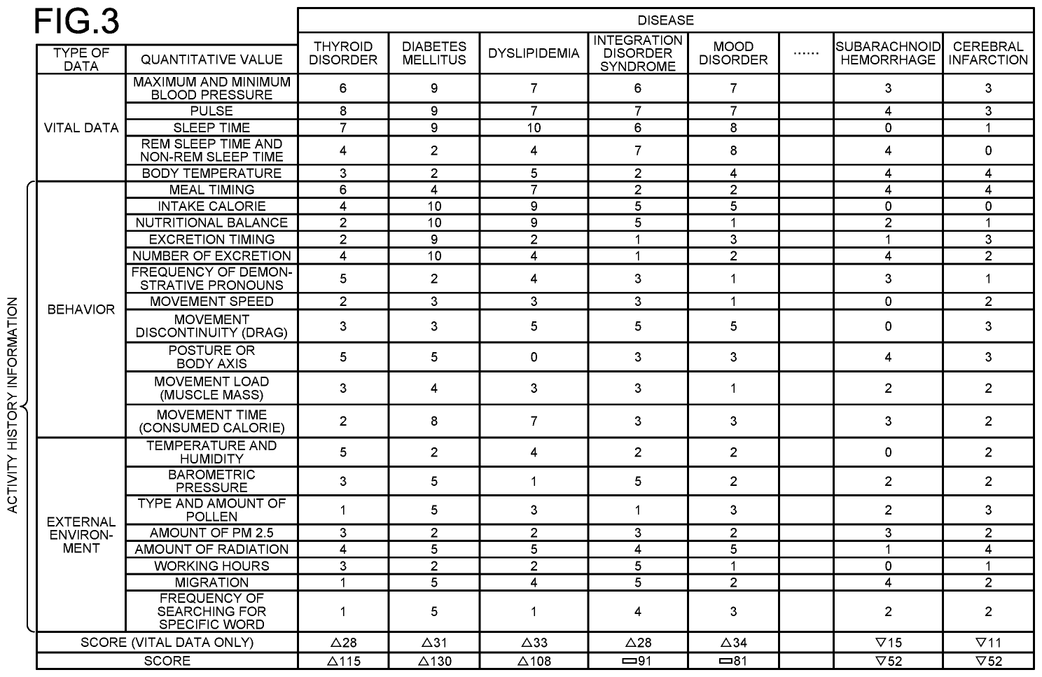

FIG. 3 is a diagram illustrating an example of disease-specific scoring according to the first embodiment. As illustrated in FIG. 3, the estimation function 152 indicates a degree of a possibility that the patient P is contracting each disease as numerical values of “0” to “10” with respect to each item included in the vital data and activity history information of the patient P. For example, in an example illustrated in FIG. 3, a score indicating a degree of a relation between maximum and minimum blood pressure values of the patient P and “thyroid disorder” is “6”. The estimation function 152 calculates a total value of the scores for each disease. In a case where the total value of the disease scores is equal to or greater than a defined threshold value, the estimation function 152 estimates the disease as a disease candidate that the patient P may contract a disease.

In FIG. 3, the estimation function 152 calculates two types of a total score based only on the vital data and a total score based on the vital data and the activity history information, but may calculate only the total score based on the vital data and the activity history information.

The total value of the scores for each disease indicates the possibility that the patient P is contracting the disease. In the present embodiment, the information indicating the degree of the possibility that the patient P is contracting each disease, such as the total value of the scores for each disease illustrated in FIG. 3, is referred to as information on a risk of contracting a disease.

In addition, in FIG. 3, the estimation function 152 obtains the degree of the possibility that the patient P is contracting each disease for each item, but the estimation function 152 may obtain the degree of the possibility that the patient P is contracting each disease with a plurality of items as AND conditions.

For example, the estimation function 152 may estimate that in a case where a temperature around the patient P, for example, a room temperature of the room 41 is equal to or higher than 28° C., and the patient P moves at equal to or higher than a defined load, a degree of a possibility that the patient P is contracting a disease called “heat stroke” is higher than that in a case where the room temperature of the room 41 is lower than 28° C. or the patient P does not move at equal to or higher than the defined load. In addition, as another example, the estimation function 152 may determine that in a case where an altitude of a current position of the patient P is equal to or higher than a threshold value, and a pulse is equal to or greater than a threshold value or blood oxygen saturation is equal to or lower than a threshold value, the patient P is contracting a disease called “altitude sickness”. In this case, since the altitude of the current position of the patient P is a condition, the estimation function 152 determines that in a case where the altitude of the current position of the patient P is not equal to or higher than the threshold value, the possibility of “altitude sickness” is low even though the pulse or blood oxygen saturation is the same as the above case.

In addition, the estimation function 152 may further estimate the disease candidate of the subject based on the attribute information of the patient P in addition to the vital data and the activity history information. For example, the estimation function 152 may estimate such that the degree of the probability that the patient P is contracting the disease “heat stroke” differs depending on the age of the patient P even though the same room temperature of the room 41 is applied.

The method of estimating the disease candidate illustrated in FIG. 3 is an example, and the estimation function 152 may estimate the disease candidate of the patient P using a method such as a learned model or a mathematical model. For example, the estimation function 152 may estimate the disease candidate of the patient P by a learned model in which the vital data and activity history information of the subject and a disease contracted by the subject are associated with each other. The learned model is, for example, a learned model generated by deep learning such as a neural network. As a deep learning method, a convolutional neural network (CNN), a recurrent neural network (RNN), and the like can be applied, but the present embodiment is not limited thereto.

The estimation function 152 transmits the estimated disease candidate to the notification necessity determination function 153 and the interview processing function 154. There may be a plurality of disease candidates.

Returning to FIG. 1, the notification necessity determination function 153 determines necessity of a notification to the patient P based on the disease candidate estimated by the estimation function 152 and the information on a risk of contracting a disease for each disease candidate. For example, the notification necessity determination function 153 may determine that a notification to the patient P is always required in a case where there is a disease candidate that the patient P is likely to contract.

Alternatively, the notification necessity determination function 153 may determine that a notification to the patient P is required in a case where a specific disease is included in the disease candidate. The specific disease that requires a notification includes, for example, a case where the patient P oneself may not be aware of contracting a disease, such as heat stroke and altitude sickness, a case of requiring urgent action, or the like. In this case, for example, the storage 120 stores notification necessity information in which necessity of a notification for each disease and contents of the notification are associated with each other, and the notification necessity determination function 153 may determine the necessity of a notification to the patient P based on the notification necessity information. Criteria for the necessity of the notification are not limited thereto.

In a case where the notification necessity determination function 153 determines that the notification to the patient P is required, the notification necessity determination function 153 transmits a disease candidate of the notification subject to the output control function 155.

The interview processing function 154 determines the necessity of interviewing the patient P based on the disease candidate estimated by the estimation function 152 and the information on a risk of contracting a disease for each disease candidate. For example, the interview processing function 154 determines that an interview for determining a disease of the subject is required in a case where there are a plurality of disease candidates.

Alternatively, the interview processing function 154 may determine that an interview with the patient P is required in a case where a specific disease is included in the disease candidate. The specific disease that requires an interview includes, for example, a disease that requires urgent response or may be aggravated, and a disease that is difficult to be identified only from the vital data and activity history information or has a characteristic subjective symptom, and the like. In this case, for example, the storage 120 stores interview information in which necessity of an interview for each disease and inquiry items in the interview are associated with each other, and the interview processing function 154 may determine the necessity of interviewing the patient P based on the interview information. Criteria for the necessity of the interview are not limited thereto.

In a case where the interview processing function 154 determines to require an interview with the patient P, the interview processing function 154 determines interview items for determining a disease of the patient P from the disease candidates.

The interview items are inquiry items for further narrowing down the disease candidates estimated by the estimation function 152, and differ depending on the estimated disease candidates. For example, the interview processing function 154 extracts interview items related to the estimated disease candidates from a plurality of the interview items stored in advance in the storage 120. In addition, the interview processing function 154 excludes symptoms that can be identified from already acquired information such as the vital data and the activity history information from the interview items.

In a case where the interview processing function 154 determines to require the interview with the patient P, the interview processing function 154 transmits the determined interview items to the output control function 155.

In addition, the interview processing function 154 identifies a disease that is most likely to be contracted by the patient P among the disease candidates, based on responses to the interview, which are input by the patient P. The interview processing function 154 transmits the identified disease to the output control function 155.

The output control function 155 causes the output unit to output a notification regarding the disease candidates estimated by the estimation function 152 or interview items. In the present embodiment, the output control function 155 causes the mobile terminal 200 held by the patient P to output the notification regarding the disease candidates estimated by the estimation function 152 or the interview items for determining a disease of the subject from the disease candidates. The output control function 155 causes the display 240 of the mobile terminal 200 to display an interview screen including the notification or the interview items by transmitting, for example, the contents of the notification or the interview items and a control signal to the mobile terminal 200.

The contents of the notification may be, for example, a message indicating a disease candidate such as “There is a possibility of heat stroke” or a message instructing a response to the disease candidate such as “Drink water”. In this case, the output control function 155 may cause the mobile terminal 200 to display a recommended examination according to the disease candidate.

In addition, the output control function 155 causes the mobile terminal 200 held by the patient P to output the disease that is identified by the interview processing function 154 and most likely to be contracted by the patient P.

Next, a flow of processing executed by the health management apparatus 100 configured as described above will be described.

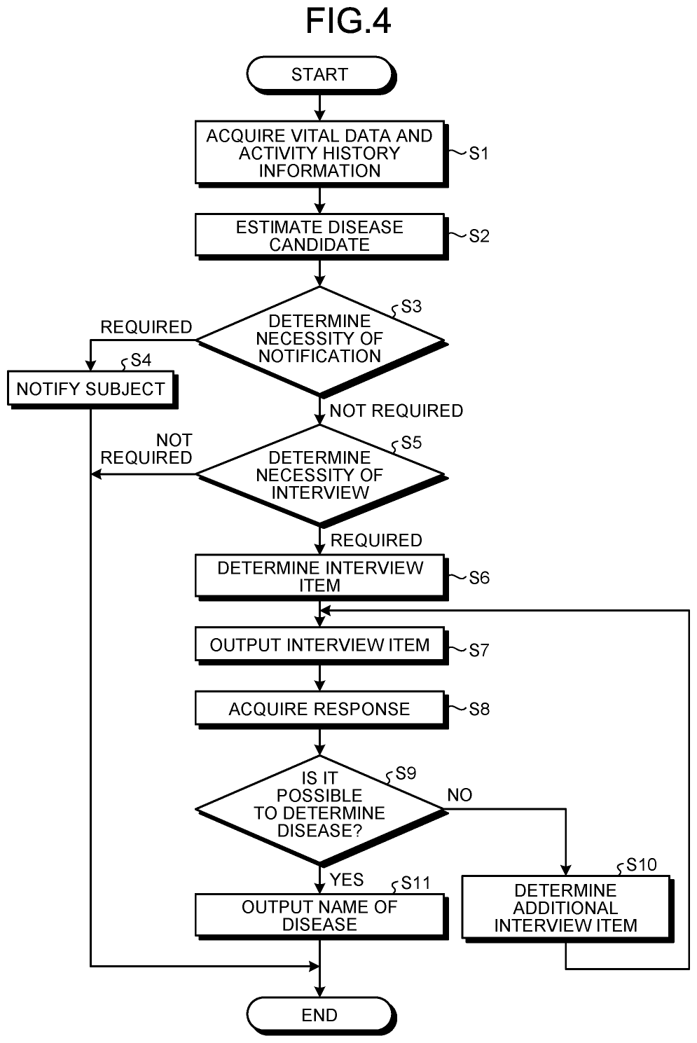

FIG. 4 is a flowchart illustrating an example of a flow of health management processing executed by the health management apparatus 100 according to the first embodiment. The processing of this flowchart is repeatedly executed, for example, at regular intervals. Alternatively, the processing of this flowchart may be executed at a timing when the mobile terminal 200 collects data of the subject.

First, the acquisition function 151 acquires vital data and activity history information of the patient P (S1).

Next, the estimation function 152 estimates disease candidates of the patient P who is a subject, based on the acquired vital data and activity history information (S2).

Then, the notification necessity determination function 153 determines necessity of a notification to the patient P based on the disease candidates estimated by the estimation function 152 and information on a risk of contracting a disease for each disease candidate (S3).

In a case where the notification necessity determination function 153 determines to require the notification to the patient P (“Required” at S3), the output control function 155 notifies the patient P of information on the disease candidates (S4). Specifically, the output control function 155 transmits a notification regarding the disease candidates estimated by the estimation function 152 and a control signal to the mobile terminal 200 held by the patient P, thereby causing the mobile terminal 200 to display the notification. For example, the receiving function 253 of the mobile terminal 200 receives the notification from the health management apparatus 100 through the NW interface 210. The display control function 254 of the mobile terminal 200 displays the notification on the display 240.

In addition, in a case where the notification necessity determination function 153 determines that the notification to the patient P is not required (“Not required” at S3), the interview processing function 154 determines necessity of interviewing the patient P based on the disease candidates estimated by the estimation function 152 and the information on a risk of contracting a disease for each disease candidate (S5).

In a case where the interview processing function 154 determines to require an interview with the patient P (“Required” at S5), the interview processing function 154 determines interview items for determining a disease that the patient P is contracting among the disease candidates (S6).

Then, the output control function 155 transmits the interview items determined by the interview processing function 154 to the mobile terminal 200 held by the patient P who is a subject (S7). As a result, the output control function 155 causes the mobile terminal 200 to display an interview screen including the interview items.

Then, the acquisition function 151 of the health management apparatus 100 acquires responses to the interview items input by the patient P from the mobile terminal 200 (S8).

Then, the interview processing function 154 determines a disease that the patient P is contracting among the disease candidates based on the responses of the patient P (S9).

In a case where the interview processing function 154 cannot determine a disease from the disease candidates based on the acquired responses (“No” at S9), the interview processing function 154 determines an additional interview item (S10). Then, returning to the processing of S7, the processing of S7 to S10 is repeated until a disease can be determined from the disease candidates.

In a case where the interview processing function 154 can determine a disease from the disease candidates based on the acquired responses (“Yes” at S9), the interview processing function 154 transmits a name of the determined disease to the output control function 155. In this case, the output control function 155 transmits the name of the determined disease to the mobile terminal 200 held by the patient P who is a subject (S11). As a result, the output control function 155 causes the mobile terminal 200 to display the name of the disease. In addition, in this case, the output control function 155 may display a recommended examination according to the disease on the mobile terminal 200.

For example, the receiving function 253 of the mobile terminal 200 receives the name of the disease from the health management apparatus 100 through the NW interface 210. The display control function 254 of the mobile terminal 200 displays the name of the disease on the display 240.

In addition, the case where the interview processing function 154 can determine the disease from the disease candidates is, for example, a case where a score indicating a possibility that the patient P is contracting any one disease candidate among a plurality of the disease candidates is higher than a score indicating a possibility that the patient P is contracting the other disease candidate by at least a defined threshold value. Criteria for whether or not the disease can be determined are not limited thereto.

In addition, in a case where the interview processing function 154 determines that the interview with the patient P is not required (“Not required” at S5), the interview processing function 154 ends the processing of this flowchart.

In the flowchart of FIG. 4, after processing the output of the notification at S4, the processing of S5 is not executed, and the processing ends. The processing of determining the necessity of the interview at S5 may be executed regardless of the presence or absence of the necessity of the notification.

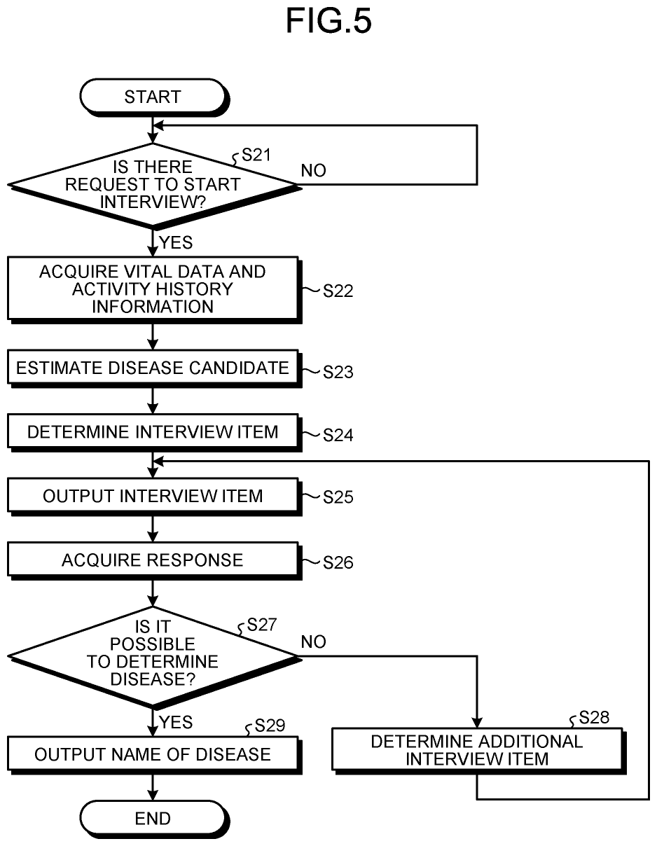

In addition, in the flowchart of FIG. 4, the processing is executed with the acquisition of the vital data and activity history information as a trigger, and the processing may be executed with a request to start the interview by the patient P as a trigger. The flow of the processing triggered by a request to start the interview by the patient P will be described with reference to FIG. 5.

FIG. 5 is a flowchart illustrating another example of a flow of the health management processing executed by the health management apparatus 100 according to the first embodiment.

In a case where the acquisition function 151 of the health management apparatus 100 acquires the request to start the interview by the patient P (“Yes” at S21), the acquisition function 151 acquires the vital data and activity history information of the patient P (S22). The request to start the interview by the patient P is received by, for example, the reception function 256 of the mobile terminal 200. Hereinafter, the processing of S23 to S29 illustrated in FIG. 5 is the same processing as S2, and S6 to S11 described with reference to FIG. 4.

In addition, in a case where the acquisition function 151 of the health management apparatus 100 does not acquire the request to start the interview by the patient P (“No” at S21), the processing of S21 is repeated and in standby status.

As described above, the health management apparatus 100 of the present embodiment estimates the disease candidates of the subject based on the vital data and activity history information of the subject, and causes the mobile terminal 200 to output the notification regarding the estimated disease candidates or the interview items for determining the disease of the subject from the disease candidates. In addition, in the present embodiment, the activity history information includes at least information on a temperature of a space around the subject, information on a movement history of the subject, and a history of position information of the subject. Therefore, with the health management apparatus 100 of the present embodiment, it is possible to effectively support the management of a health condition of the subject based on personal information of the subject.

For example, in the health management apparatus 100 of the present embodiment, even though the subject is not aware of contracting a disease, it is possible to encourage to take an action by calling attention before aggravating the disease, by the notification regarding the disease candidates estimated from the vital data and activity history information of the subject. Alternatively, in the health management apparatus 100 of the present embodiment, it is possible to reduce the number of interview items as compared with the case of estimating the disease only by the interview, by outputting the interview items for determining the disease of the subject from the disease candidates estimated in advance from the vital data and activity history information of the subject, and the time and effort for the subject to response the interview can be reduced.

In addition, in the present embodiment, the mobile terminal 200 held by the patient P who is a health management subject is an example of the output unit, and the health management apparatus 100 of the present embodiment causes the mobile terminal 200 to output the notification or the interview items, thereby presenting the notification or the interview items to the patient P. Therefore, with the health management apparatus 100 of the present embodiment, in a case where the health management subject is the same person as the user, the notification or the interview can be rapidly performed on the health management subject.

In addition, in the present embodiment, the activity history information includes a dosing history or a medication history of the subject. Therefore, with the health management apparatus 100, for example, in a case where a rash occurs on the body of the patient P and the patient P requests to start an interview, the health management apparatus 100 can determine whether or not the rash of the patient P may be a drug eruption caused by a drug taken by the patient P or a drug administered to the patient P, based on the activity history information.

### Second Embodiment

In the first embodiment described above, the health management apparatus 100 estimates the disease candidates that are likely to be contracted by the subject, but in the present embodiment, a risk of aggravation in a case where the subject has contracted each disease candidate is further estimated.

The health management system S1 of the present embodiment is provided with the health management apparatus 100 and the mobile terminal 200 in the same manner as in the first embodiment. In addition, configurations of the health management apparatus 100, the mobile terminal 200, and the wearable terminal 201 of the present embodiment are the same as those of the first embodiment.

The processing circuit 150 of the health management apparatus 100 of the present embodiment is provided with the acquisition function 151, the estimation function 152, the notification necessity determination function 153, the interview processing function 154, and the output control function 155 in the same manner as in the first embodiment.

In addition to the same functions as in the first embodiment, the estimation function 152 of the present embodiment estimates a risk of aggravation of each disease candidate based on the estimated disease candidates, the vital data of the subject, the activity history information of the subject, and the attribute information of the subject.

The risk of aggravation of a disease is a possibility of aggravation in a case where the subject has contracted the disease. The estimation function 152 estimates that the higher the possibility of aggravation of the disease included in the disease candidates, the higher the risk of aggravation.

By referring to aggravation information in which a plurality of diseases are associated with characteristics of vital data, activity history information, and attribute information, in which each disease included in the diseases is likely to be aggravated, the estimation function 152 estimates that a risk of aggravation is high in a case where the vital data, the activity history information, and the attribute information of the patient P correspond to the characteristics included in aggravation information.

The aggravation information is stored in, for example, the storage 120. An administrator may register and update the aggravation information based on information on a condition of the patient diagnosed in the past, or information such as “People who develop YY symptoms are likely to be aggravated” announced at academic conferences.

The estimation function 152 determines that, for example, in a case where a value of a certain item included in the vital data is equal to or higher than a threshold value, a risk of aggravation of the certain disease increases. The risk of aggravation may be defined numerically such as a score, or may be gradually represented as levels at high, medium, and low.

In addition, the aggravation information may include not only the characteristics of the vital data, the activity history, and the attribute information, but also subjective symptoms in each disease being likely to be aggravated.

In addition, the notification necessity determination function 153 of the present embodiment may use the risk of aggravation as a criterion for determining necessity of a notification, in addition to the same functions as those of the first embodiment. For example, the notification necessity determination function 153 may determine to require a notification in a case of a disease having a higher risk of aggravation than defined criteria among diseases included in the disease candidates.

In addition, the output control function 155 of the present embodiment changes contents of the notification according to the estimated degree of the risk of aggravation, in addition to the same functions as those of the first embodiment. For example, the output control function 155 transmits the contents of the notification and a control signal to the mobile terminal 200 so as to output a message having a higher degree of warning as the risk of aggravation increases. In addition, in a case where the risk of aggravation is equal to or higher than the defined criteria, the contents of the notification may be contents in which immediate consultation with a medical institution is recommended. In addition, in a case where the risk of aggravation is lower than the defined criteria, the contents of the notification may be contents in which symptomatic treatment is recommended. Alternatively, the output control function 155 may cause the estimated degree of the risk of aggravation to be included in the contents of the notification.

In addition, the interview processing function 154 of the present embodiment may obtain a more detailed risk of aggravation of a disease narrowed down from the disease candidates, in addition to the same functions as those of the first embodiment. For example, the interview processing function 154 obtains a more detailed risk of aggravation of a disease narrowed down from the disease candidates, based on the responses of the patient P who is the subject to the interview, the vital data of the subject, the activity history information of the subject, and the attribute information of the subject.

As described above, the health management apparatus 100 of the present embodiment estimates a risk of aggravation of a disease candidate based on the estimated disease candidates, the vital data, the activity history information, and the attribute information of the subject, and changes the contents of the notification according to the degree of the risk of aggravation. Therefore, with the health management apparatus 100 of the present embodiment, it is possible to present the disease candidate and the risk of aggravation to the subject and call attention more specifically, in addition to the effect of the first embodiment.

### Third Embodiment

In the first and second embodiments described above, the example in which the health management subject is the same as the person who receives the notification regarding the disease of the health management subject is described, but in a third embodiment, in addition to the health management subject, a person other than the health management subject also receives the notification.

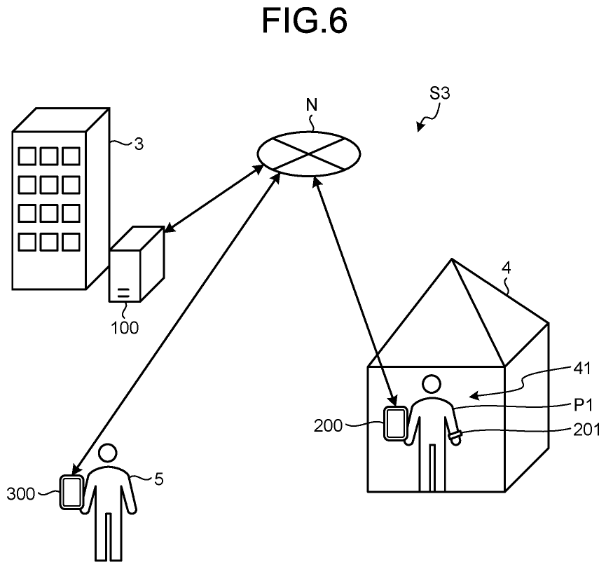

FIG. 6 is a diagram illustrating an example of a usage form of a health management system S3 according to the third embodiment. As illustrated in FIG. 6, the health management system S3 of the present embodiment is provided with the health management apparatus 100, the mobile terminal 200, and a mobile terminal 300. In addition, configurations of the health management apparatus 100 and the wearable terminal 201 of the present embodiment are the same as those of the first embodiment.

Hardware configurations of the mobile terminal 200 and the mobile terminal 300 are the same as that of the mobile terminal 200 of the first embodiment described with reference to FIG. 1. The mobile terminal 200 may be referred to as a first terminal, and the mobile terminal 300 may be referred to as a second terminal.

In the present embodiment, the mobile terminal 200 is held by the subject patient P in the same manner as the mobile terminal 200 of the first embodiment described with reference to FIG. 1.

In addition, the mobile terminal 300 is held by a related person 5 of the patient P. The related person 5 is, for example, a guardian or a relative of the patient P. In a case where the patient P is a child, the related person 5 may be a parent of the patient P. In addition, in a case where the patient P is an elderly person, the related person 5 may be a child of the patient P.

The mobile terminal 200 may be referred to as a first terminal, and the mobile terminal 300 may be referred to as a second terminal. In addition, the mobile terminals 200 and 300 are both examples of a display unit or an output unit, and a terminal in the present embodiment. Alternatively, the display 240 in each of the mobile terminals 200 and 300 may be an example of the display unit or the output unit.

The processing circuit 150 of the health management apparatus 100 of the present embodiment is provided with the acquisition function 151, the estimation function 152, the notification necessity determination function 153, the interview processing function 154, and the output control function 155 in the same manner as in the first embodiment.

The acquisition function 151 has the same function as that of the first embodiment. An acquisition source of the vital data and activity history information of the subject is the mobile terminal 200.

In addition, the estimation function 152, the notification necessity determination function 153, and the interview processing function 154 have the same functions as those of the first embodiment.

In addition to the function of the first embodiment, the output control function 155 causes the mobile terminal 300 to output the notification regarding the disease candidate of the patient P. For example, in the same manner as in the first embodiment, in a case where the output control function 155 causes the mobile terminal 200 of the patient P to output a notification, the output control function 155 causes the mobile terminal 300 held by the related person 5 to output the fact that the notification is performed with respect to the patient P and a notification of the contents notified of the patient P.

In addition, the output control function 155 may only cause the mobile terminal 300 held by the related person 5 to output a notification. For example, in a case where the health management subject is an animal such as a pet instead of a person and the related person 5 is the owner of the health management subject, the health management apparatus 100 outputs a notification regarding a disease of the health management subject only to the mobile terminal 300 held by the related person 5. In this case, the health management subject may not hold the mobile terminal 200, and the wearable terminal 201 may transmit vital data and activity history information of the subject to the health management apparatus 100 instead of the mobile terminal 200.

In addition, in a case where the output control function 155 causes the mobile terminal 300 of the related person 5 to output a notification, the output control function 155 may output the notification together with position information of the subject.

As described above, with the health management apparatus 100 of the present embodiment, in addition to the effect of the first embodiment, a notification regarding the disease candidates of the subject is output by the mobile terminal 300 held by the related person 5 of the subject, and thereby the related person 5 is capable of recognizing a health condition of the subject. Therefore, for example, even though it is difficult that the subject appropriately carries out one's own health management, the related person 5 may contact the subject or the related person 5 may rush to a location where the subject is present and respond.

### Fourth Embodiment

In the first to third embodiments described above, the example in which the disease candidates are estimated based on the information of the health management subject oneself is described, but in a fourth embodiment, information on a third party is used to estimate the disease candidates of the health management subject.

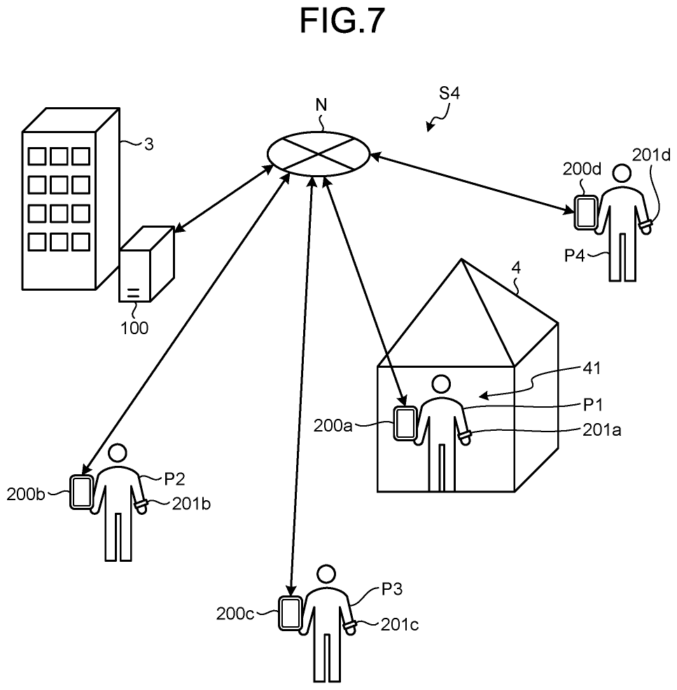

FIG. 7 is a diagram illustrating an example of a usage form of a health management system S4 according to the fourth embodiment. As illustrated in FIG. 7, the health management system S4 of the present embodiment is provided with the health management apparatus 100, and a plurality of mobile terminals 200a to 200d. The mobile terminals 200a to 200d are held by patients P1 to P4, which are health management subjects, respectively. In a case where the patients P1 to P4 are not particularly distinguished, the patients P1 to P4 are simply referred to as a patient P. For one patient P, the other patient P is a third party.

In addition, the mobile terminals 200a to 200d are connected in a communicable manner to wearable terminals 201a to 201d attached to the patients P1 to P4, respectively. The number of patients P1 to P4, the number of mobile terminals 200a to 200d, and the number of wearable terminals 201a to 201d are not limited to the example illustrated in FIG. 7.

In addition, in a case where the mobile terminals 200a to 200d are not particularly distinguished, the mobile terminals 200a to 200d are simply referred to as a mobile terminal 200. In addition, in a case where the wearable terminals 201a to 201d are not particularly distinguished, the wearable terminals 201a to 201d are simply referred to as a wearable terminal 201.

A configuration of the wearable terminal 201 is the same as that of the first embodiment.

A configuration of the health management apparatus 100 is the same as that of the first embodiment. In addition, hardware configurations of the mobile terminals 200a to 200d are the same as that of the mobile terminal 200 in the first embodiment described with reference to FIG. 1. In addition, hardware configurations of the wearable terminals 201a to 201d are the same as that of the wearable terminal 201 in the first embodiment described with reference to FIG. 1.

The processing circuit 150 of the health management apparatus 100 of the present embodiment is provided with the acquisition function 151, the estimation function 152, the notification necessity determination function 153, the interview processing function 154, and the output control function 155 in the same manner as in the first embodiment. The notification necessity determination function 153 has the same function as that of the first embodiment.

The acquisition function 151 has the same function as that of the first embodiment. In addition, the acquisition function 151 of the present embodiment acquires vital data of each of the patients P1 to P4 and activity history information of each of the patients P1 to P4 from each of the mobile terminals 200a to 200d. In addition, in the acquisition function 151, the frequency of the acquisition of the vital data and activity history information of the subject increases as a degree of an infection risk of the subject estimated by the estimation function 152 described later increases.

The estimation function 152 has the same function as that of the first embodiment. In addition, the estimation function 152 of the present embodiment estimates the disease candidates of the patient P based on information on a disease of the third party in addition to the vital data and activity history information of the patients P. The estimation function 152 estimates that in a case where the illness contracted by the third party is an infectious illness and the subject is a close contact with the third party, the subject is likely to be infected with the infectious illness contracted by the third party.

The information on the disease of the third party includes at least a name of the illness contracted by the third party and position information of the third party. In addition, the information on the disease of the third party may include not only the disease that has been confirmed to be contracted by the third party but also names of diseases included in the disease candidates. The third party in the present embodiment is referred to as another patient P. Specifically, in a case where the patient P1 is a subject, the information on the disease of the third party is information on disease candidates estimated by the estimation function 152 with respect to the other patients P2 to P4, or information on diseases identified by the interview processing function 154 with respect to the other patients P2 to P4.

Regarding the infectious illness among diseases, an exposure history is important in determining the infection risk. The exposure history is experience of being exposed to problematic risk factors (absorption, inhalation, contact, and the like), such as close contact with an infected person. For example, in a case where the patient P2 is contracting an infectious illness, a contact between the patient P1 and the patient P2 is one of grounds for determining whether or not the patient P1 is contracting the infectious illness.

Therefore, the estimation function 152 estimates a degree of a possibility that the patient P1 contracts the infectious illness, that is a degree of an infection risk based on vital data and activity history information of the patient P1 and information on a disease of the third party. For example, the estimation function 152 estimates whether or not the patient P1 is in close contact with the patient P2, or a length of the contact time between the patient P1 and the patient P2 based on past position information of the patient P1 and past position information of the other patient P2 who has contracted the infectious illness, and estimates a possibility that the patient P1 is contracting the infectious illness based on a lower order estimation result. In addition, the estimation function 152 may estimate a possibility that the patient P1 is contracting the infectious illness by identifying vaccination of the patient P1 from a medication history included in activity history information of the patient P1.

In addition, the information on the disease of the third party is not limited to the disease candidates estimated by the estimation function 152 or the diseases identified by the interview processing function 154. The information on the disease of the third party may include countries or regions where an infectious illness is endemic, an outbreak location of mass infection such as clusters and an outbreak time, and movement records of infected persons. In this case, the third party is not limited to the patient holding the mobile terminal 200. For example, the acquisition function 151 may acquire information on the disease of the third party from countries, local public bodies, medical institutions, or the like.

In addition, since the infectivity and a way of infection differ depending on types of infectious illnesses, the estimation function 152 may estimate a possibility that the patient P1 is contracting the infectious illness according to a type of the infectious illness that the other patient P2 has contracted. The way of infection includes contact infection, airborne infection, droplet infection, mediator infection, and the like.

In addition, the estimation function 152 may estimate the infectivity of the patient P2 at that time from a symptom and a virus content of the other patient P2 at the time when coming into contact with the patient P1. The virus content is estimated based on, for example, a length of a period since the other patient P2 has contracted the infectious illness or a length of the time after a symptom appears. The symptom of the other patient P2 is, for example, the presence or absence of a cough, fever symptom, or the like.

In addition, the estimation function 152 may estimate a degree of the infection risk according to a length of a distance from an outbreak location of mass infection of the infectious illness to a position of the patient P1 within a defined period before and after the outbreak of the mass infection. For example, in a case where before and after the time when a cluster occurs, the patient P1 was present within the defined distance from the center of the cluster occurred, the estimation function 152 estimates that the patient P1 has a high risk of infection.

In addition, the estimation function 152 may comprehensively obtain a degree of the possibility that the patient P1 is contracting the infectious illness, that is, a risk of contracting a disease from a plurality of items included in the vital data of the patient P1, the activity history information of the patient P1, and the information on the disease of the third party. In the risk of contracting a disease, a risk of the infectious illness is specifically referred to as an infection risk.

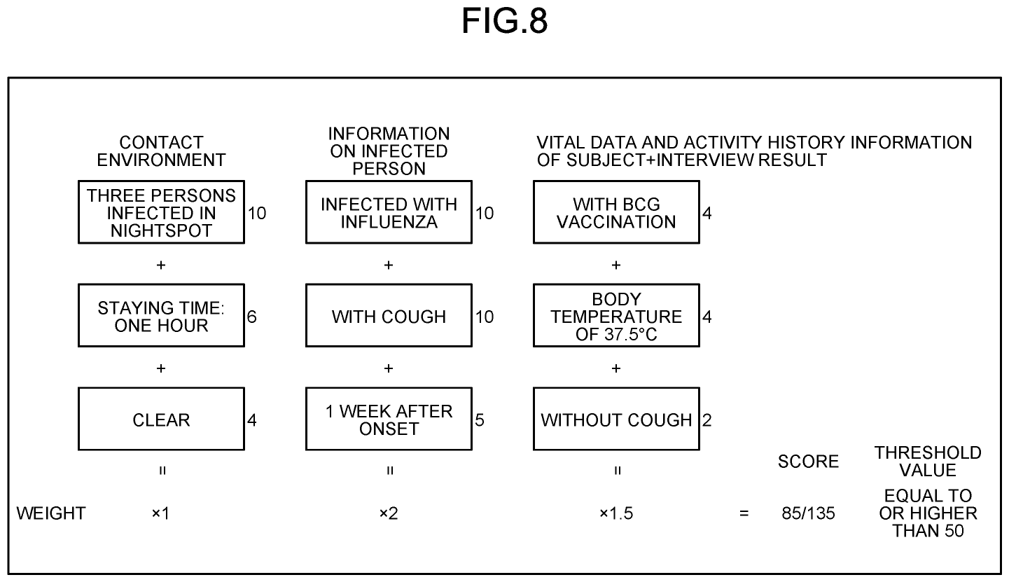

FIG. 8 is a diagram illustrating an example of a method of estimating the risk of contracting a disease according to the fourth embodiment. As illustrated in FIG. 8, the estimation function 152 obtains a score indicating a degree of the possibility that the patient P1 is contracting the infectious illness based on a contact environment of the patient P1, information on an infected person, the vital data and activity history information of the patient P1 who is a subject, which are included in the items included in the vital data of the patient P1, the activity history information of the patient P1, and the information on the disease of the third party, and the interview result of the patient P1. The estimation function 152 obtains a total score obtained by weighting and adding the score of each item for each classification as a numerical value indicating the degree of the risk of contracting a disease. It is assumed that the score of each item and a value of a weighting coefficient for each classification are stored in, for example, the storage 120.

In addition, the estimation function 152 determines that the patient P1 has a possibility to contract the infectious illness in a case where the numerical value indicating the degree of the risk of contracting a disease is equal to or higher than a threshold value.

In addition, the output control function 155 is provided with the same function as that of the first embodiment and causes the output unit to output a notification regarding the infectious illness estimated such that the subject is likely to be contracted. For example, in a case where the estimation function 152 estimates that the patient P1 is likely to contract the infectious illness, the output control function 155 causes the mobile terminal 200a of the patient P1 to output the notification regarding the infectious illness. In addition, the output control function 155 may cause information on recommended examinations to be included in the contents of the notification in a case where it is estimated that the subject is likely to contract the infectious illness.

In addition, the interview processing function 154 has the same function as that of the first embodiment.

Specifically, in a case where the necessity of interviewing the patient P is determined based on the disease candidates estimated by the estimation function 152 and the information on the risk of contracting a disease for each disease candidate, and it is determined to require the interview, interview items for determining a disease of the patient P is determined from the disease candidates.

For example, in the present embodiment, the estimation function 152 estimates the infection risk of the infectious illness based on the information on the disease of the third party, but the interview processing function 154 determines interview items for improving the accuracy of the diagnosis of the infectious illness in a case where the infection risk of the infectious illness is equal to or higher than defined criteria. For example, in a case where the infection risk of the infectious illness is high, the interview processing function 154 causes inquiries regarding the presence or absence of symptoms of the infectious illness with which the subject may be infected, for example, a respiratory symptom, and a degree thereof to be included in the interview items. In addition, in a case where a plurality of infectious illnesses are included in the disease candidates, the estimation function 152 causes an inquiry regarding a symptom of each infectious illness to be included in the interview items.

In addition, in a case where the infection risk of the infectious illness is low, the interview processing function 154 determines interview items for narrowing down a disease that the subject is contracting from the disease candidates other than the infectious illness. For example, in a case where it is estimated that the infection risk of the infectious illness is low from an exposure history or the like even though vital data is similar to a symptom of the infectious illness, the interview processing function 154 causes inquiries regarding symptoms of the other disease to be included in the interview items.

As described above, the health management apparatus 100 of the present embodiment estimates the disease candidates of the subject based on the vital data of the subject, the activity history information of the subject, and the information on the disease of the third party. Therefore, with the health management apparatus 100 of the present embodiment, in addition to the effect of the first embodiment, it is possible to accurately determine the infectious illness that the subject is likely to contract by contacting with the infected person, among diseases.

In addition, in the present embodiment, the infectious illness is described as an example, but the configuration of the health management apparatus 100 of the present embodiment can be applied to diseases other than the infectious illness. For example, with the health management apparatus 100 of the present embodiment, in a case where heat stroke is likely to occur locally, it is possible to accurately estimate a possibility that the subject oneself has developed heat stroke according to the presence or absence of development of heat stroke of the third party around the subject by using information on the disease of the third party to estimate disease candidates.

In addition, the health management apparatus 100 of the present embodiment estimates that the subject is likely to be infected with the infectious illness that the third party has contracted in a case where an illness contracted by the third party is the infectious illness and the subject is a close contact with the third party, and causes the mobile terminal 200 held by the subject to output a notification regarding the infectious illness. Therefore, with the health management apparatus 100 of the present embodiment, even though the subject does not have any subjective symptoms, it is possible to make the subject grasp the possibility of contracting the infectious illness and encourage to take an action rapidly.

In addition, the health management apparatus 100 of the present embodiment estimates the degree of the infection risk regarding the infectious illness of the subject based on the vital data of the subject, the activity history information of the subject, and the information on the disease of the third party. Therefore, with the health management apparatus 100 of the present embodiment, it is possible to take an action rapidly with respect to the infectious illness by changing the contents of the notification, the interview items, and the like according to the risk of the infectious illness.

In addition, the health management apparatus 100 of the present embodiment increases the acquisition frequency of the vital data and the activity history information as the estimated infection risk increases. In a case where the subject is contracting the infectious illness, a condition may change rapidly over a short period of time. According to the health management apparatus 100 of the present embodiment, in a case where the infection risk of the subject is high, a change in a physical condition of the subject can be rapidly grasped by the acquisition frequency of the vital data and activity history information of the subject being increased.

In addition, in the present embodiment, the information on the disease of the third party includes information on a location where the outbreak of the mass infection of the infectious illness occurs, and the health management apparatus 100 of the present embodiment estimates the degree of the infection risk of the subject according to a length of a distance from the location where the outbreak of the mass infection of the infectious illness occurs to the subject within a defined period before and after the outbreak of the mass infection. Therefore, with the health management apparatus 100 of the present embodiment, the possibility that the subject is contracting the infectious illness can be estimated with high accuracy.

### Fifth Embodiment

In the fourth embodiment described above, the possibility that the subject is contracting the infectious illness is estimated based on the contact between the subject and the infected person, but not only a case where the subject comes into contact with the infected person but also a case where the infected person comes into contact with the subject may also be taken into consideration as a factor of the infection risk.

The health management system S4, the health management apparatus 100, the mobile terminals 200a to 200d, and the wearable terminals 201a to 201d of the present embodiment have the same configurations as those of the fourth embodiment.

The processing circuit 150 of the health management apparatus 100 of the present embodiment is provided with the acquisition function 151, the estimation function 152, the notification necessity determination function 153, the interview processing function 154, and the output control function 155 in the same manner as in the fourth embodiment.

In the present embodiment, an infection preliminary group includes people who are close contacts with the infected person. More specifically, those who are close contacts with those who have been confirmed to be infected are defined as a first infection preliminary group. In addition, a person who is a close contact with the person included in the first infection preliminary group is defined as a second infection preliminary group.

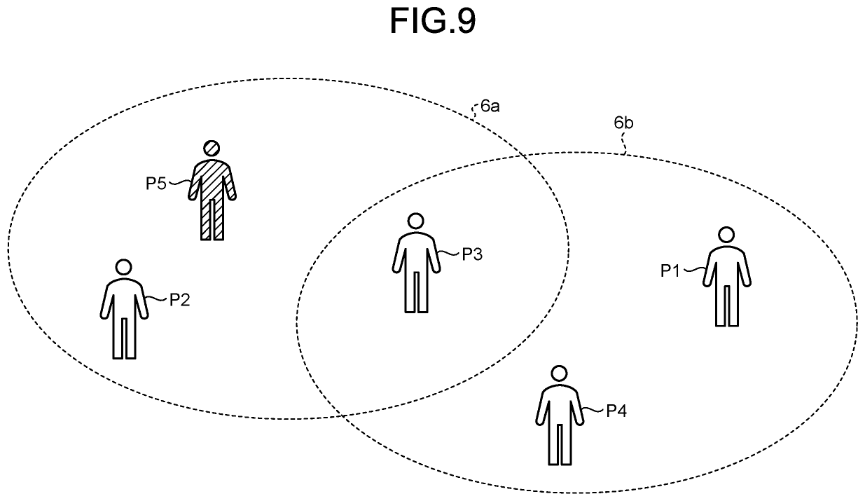

FIG. 9 is a diagram illustrating an example of an infection preliminary group according to a fifth embodiment. In the example illustrated in FIG. 9, it is assumed that the patient P5 is an infected person, for example. In the example illustrated in FIG. 9, it is assumed that the patient P5 is an infected person and the other patients P1 to P4 have not yet been turned out to be infected. The patients P2 and P3 are close contacts with the patient P5. In addition, the patients P1 and P4 are close contacts with the patient P3. In this case, the patients P2 and P3 are included in a first infection preliminary group 6a. In addition, the patients P1 and P4 are included in a second infection preliminary group 6b.

The estimation function 152 of the present embodiment estimates that in a case where an illness contracted by the third party is an infectious illness, a person who is a close contact with the third party is included in the first infection preliminary group 6a, and a person who is a close contact with the person included in the first infection preliminary group is included in the second infection preliminary group 6b. More specifically, the estimation function 152 determines the presence or absence of close contact of each of the patients P1 to P5 from position information of the patients P1 to P5 acquired by the acquisition function 151. The presence or absence of close contact is determined by, for example, the closest distance between the patients P1 to P5 and a length of a time when the close distance is maintained. In a case where any of the patients P1 to P5 is infected with the infectious illness, the estimation function 152 estimates the first infection preliminary group 6a and the second infection preliminary group 6b based on an infected person.

In addition to the same functions as those of the fourth embodiment, the notification necessity determination function 153 of the present embodiment determines that the notification is required in a case where the subject is included in the first infection preliminary group 6a. In addition, in a case where it is turned out that the person included in the first infection preliminary group 6a has contracted the infectious illness, it is determined that the notification is required in a case where the subject is included in the second infection preliminary group 6b. Specifically, in the example illustrated in FIG. 9, in a case where it is turned out that the patient P5 has contracted the infectious illness, the notification necessity determination function 153 determines that the patients P2 and P3 are required to be notified. In addition, in a case where it is turned out that the patient P3 has contracted the infectious illness, the notification necessity determination function 153 determines that the patients P1 and P4 are required to be notified.

In addition, the output control function 155 of the present embodiment causes the output unit held by the subject to output the notification according to the determination result of the notification necessity determination function 153. For example, the output control function 155 of the present embodiment causes the mobile terminal 200 held by the subject to output the notification regarding the infectious illness estimated such that the subject is likely to be contracted in a case where the subject is included in the first infection preliminary group 6a.

In addition, in a case where it is turned out that the person included in the first infection preliminary group 6a has contracted the infectious illness and a case where the subject is included in the second infection preliminary group 6b, the output control function 155 of the present embodiment causes the mobile terminal 200 held by the subject to output the notification regarding the infectious illness estimated such that the subject is likely to be contracted.

As described above, with the health management apparatus 100 of the present embodiment, not only the first infection preliminary group 6a in direct contact with the infected person but also the second infection preliminary group 6b in indirect contact with the infected person through the first infection preliminary group 6a are estimated in advance, so that in a case where it is turned out that the person included in the first infection preliminary group 6a is infected, the subject who has contacted with this person can be rapidly notified.

### Sixth Embodiment

In the above described first to fifth embodiments, the interview screen is displayed on the mobile terminal 200 held by the subject, but the interview screen may be displayed on a terminal used by a doctor.

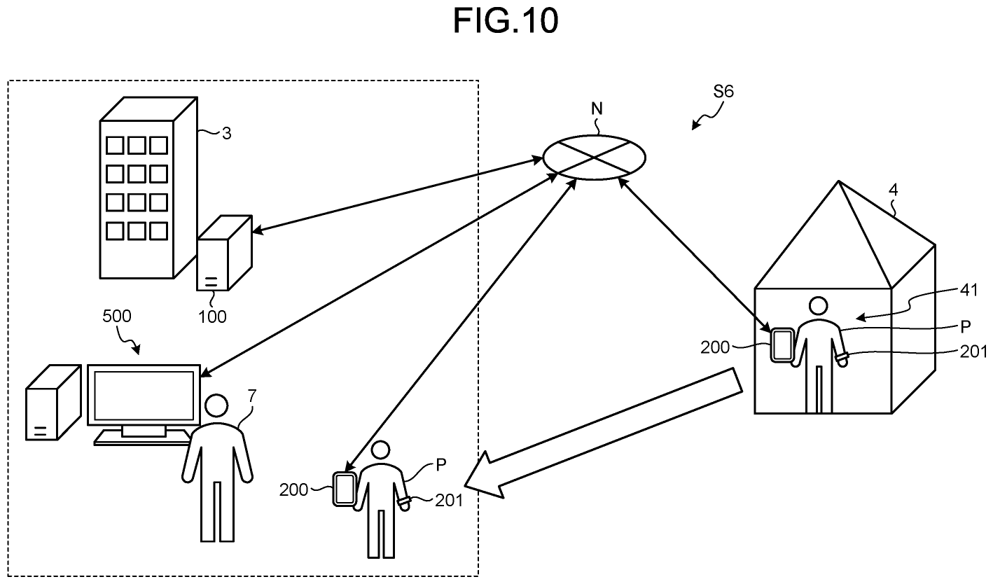

FIG. 10 is a diagram illustrating an example of a usage form of a health management system S6 according to a sixth embodiment. In the present embodiment, the health management system S6 functions as an interview support system that supports an interview with a doctor 7.

The health management system S6 of the present embodiment is provided with the health management apparatus 100, the mobile terminal 200, and an interview terminal 500.

The interview terminal 500 is a PC or a tablet terminal used by the doctor 7 to interview the patient P. A hardware configuration of the interview terminal 500 is the same as that of the health management apparatus 100. In addition, in the present embodiment, the interview processing function 154 in the above described first to fifth embodiments is executed by a processing circuit of the interview terminal 500. Alternatively, in the present embodiment, the interview terminal 500 may display disease candidates so that the doctor 7 can grasp a disease, and the doctor 7 may determine the contents of the interview for identifying the disease from the disease candidates. In addition, in the present embodiment, the entire interview terminal 500 or a display of the interview terminal 500 is an example of the output unit.

For example, in the example illustrated in FIG. 10, the health management apparatus 100 and the interview terminal 500 are provided in, for example, the medical institution 3. In a case where the patient P visits the medical institution 3 to receive a diagnosis of the doctor 7, the estimation function 152 of the health management apparatus 100 estimates the disease candidates from the vital data and the activity history information of the patient P acquired from the mobile terminal 200. The output control function 155 can support the doctor 7 to interview the patient P by causing the interview terminal 500 to output the disease candidates.

The health management apparatus 100 and the interview terminal 500 may be realized by the same device. In addition, it is described in FIG. 10 that the patient P visits the medical institution 3, but the health management system S6 can also be applied to a remote diagnosis for the patient P who is outside the medical institution 3.

### Modified Example 1

In each of the above described embodiments, the notification and the interview items are displayed on the display, but may be output by voice.

In this case, for example, a speaker (not illustrated) of the mobile terminal 200 is an example of the output unit. In addition, in this case, the processing circuit 250 of the mobile terminal 200 includes a voice output control function instead of the display control function 254 or in addition to the display control function 254. The display control function 254 and the voice output control function may be used as an example of the output control unit.

### Modified Example 2

In each of the above described embodiments, the health management apparatus 100 estimates the disease candidates and then notifies the disease candidates, but in a case where the vital data satisfies a defined condition, the notification to the subject may be rapidly performed without the estimation processing.

For example, the output control function 155 of the health management apparatus 100 causes the mobile terminal 200 to output a notification regarding a health condition of the subject in a case where the vital data of the subject satisfies the defined condition. Examples of the defined condition include a condition that matches symptoms of an infectious illness, such as “a fever of equal to or higher than 37.5° C. continued for 2 days”. Alternatively, the defined condition may be a case where a value greater than a normal range of the vital data is measured.

### Modified Example 3

In addition, in each of the above described embodiments, the health management apparatus 100 is provided with both a function of the notification to the subject and a function of the output of the interview items, but may be provided with any one of the notification function and the interview function.

### Modified Example 4

In addition, in each of the above described embodiments, the health management apparatus 100 is installed in the medical institution 3, but the health management apparatus 100 may be provided in a cloud environment or the like. In addition, the storage 120 of the health management apparatus 100 may be provided outside the health management apparatus 100.

According to at least one embodiment described above, it is possible to support the management of the health condition of the subject.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A health management apparatus comprising:
processing circuitry configured to
acquire vital data and activity history information of a subject,
estimate a disease candidate of the subject based on the acquired vital data and the acquired activity history information, and
cause a display, a speaker, or a terminal to output a notification regarding the estimated disease candidate or an interview item for determining a disease of the subject from the disease candidate, wherein
the activity history information includes at least information on a temperature of a space around the subject, information on a movement history of the subject, and a history of position information of the subject.

2. The health management apparatus according to claim 1, wherein
the processing circuitry is configured to
acquire attribute information of the subject,
estimate a risk of aggravation of the disease candidate based on the estimated disease candidate, the vital data, the activity history information, and the attribute information of the subject, and
change contents of the notification according to a degree of the risk of aggravation.

3. The health management apparatus according to claim 1, wherein
the processing circuitry is configured to estimate a disease candidate of the subject based on the vital data, the activity history information, and information on a disease of a third party.

4. The health management apparatus according to claim 3, wherein
the information on the disease of the third party includes at least a name of an illness contracted by the third party and position information of the third party, and
the processing circuitry is configured to
estimate that in a case where the illness contracted by the third party is an infectious illness and the subject is a close contact with the third party, the subject is likely to be infected with the infectious illness contracted by the third party, and
cause the display, the speaker, or the terminal to output a notification regarding the infectious illness estimated such that the subject is likely to be contracted.

5. The health management apparatus according to claim 3, wherein
the processing circuitry is configured to
estimate that in a case where the illness contracted by the third party is an infectious illness, a person who is a close contact with the third party is included in a first infection preliminary group, and a person who is a close contact with the person included in the first infection preliminary group is included in a second infection preliminary group, and
cause the display, the speaker, or the terminal to output a notification regarding the infectious illness estimated such that the subject is likely to be contracted in a case where the person included in the first infection preliminary group is turned out to contract the infectious illness, and the subject is included in the second infection preliminary group.

6. The health management apparatus according to claim 3, wherein
the processing circuitry is configured to estimate a degree of an infection risk regarding the infectious illness of the subject based on the vital data, the activity history information, and the information on the disease of the third party.

7. The health management apparatus according to claim 6, wherein
the processing circuitry is configured to increase an acquisition frequency of the vital data and the activity history information as the estimated infection risk increases.

8. The health management apparatus according to claim 6, wherein
the information on the disease of the third party includes information on an outbreak location of mass infection of an infectious illness, and
the processing circuitry is configured to estimate a degree of the infection risk of the subject according to a length of a distance from the outbreak location to a position of the subject within a defined period before and after the outbreak of the mass infection.

9. The health management apparatus according to claim 1, wherein
the processing circuitry is configured to cause the display, the speaker, or the terminal to output a notification regarding a health condition of the subject in a case where the vital data satisfies a defined condition.

10. The health management apparatus according to claim 1, wherein
the terminal is a terminal held by the subject, and
the processing circuitry is configured to present the notification or the interview item to the subject by causing the terminal to output the notification or the interview item.

11. The health management apparatus according to claim 1, wherein
the terminal is a terminal held by a related person of the subject, and
the processing circuitry is configured to cause the terminal to output the notification regarding the disease candidate of the subject.

12. The health management apparatus according to claim 1, wherein
the processing circuitry is configured to determine a disease of the subject from the disease candidate based on a response of the subject to the interview item.

13. The health management apparatus according to claim 1, wherein
the activity history information further includes information on a dosing history or a medication history of the subject, or a medical history of the subject.

14. A health management system comprising:
at least one terminal; and
a health management apparatus, wherein
the at least one terminal includes a first processing circuitry configured to
acquire vital data and activity history information of a subject,
transmit the vital data and the activity history information to the health management apparatus, and
output a notification or an interview item output from the health management apparatus to a display or a speaker,
the health management apparatus includes a second processing circuitry configured to
acquire the vital data and the activity history information from the at least one terminal,
estimate a disease candidate of the subject based on the acquired vital data and the acquired activity history information, and
transmit a notification regarding the estimated disease candidate with respect to the subject or a related person of the subject or an interview item for determining a disease of the subject from the disease candidate to the at least one terminal, and
the activity history information includes at least information on a temperature of a space around the subject, information on a movement history of the subject, and a history of position information of the subject.

15. The health management system according to claim 14, wherein
the second processing circuitry is configured to
acquire attribute information of the subject,
estimate a risk of aggravation of the disease candidate based on the estimated disease candidate, the vital data, the activity history information, and the attribute information of the subject, and
change contents of the notification according to a degree of the risk of aggravation.

16. The health management system according to claim 14, wherein
the second processing circuitry is configured to estimate a disease candidate of the subject based on the vital data, the activity history information, and information on a disease of a third party.

17. The health management system according to claim 16, wherein
the information on the disease of the third party includes at least a name of an illness contracted by the third party and position information of the third party, and
the second processing circuitry is configured to
estimate that in a case where the illness contracted by the third party is an infectious illness and the subject is a close contact with the third party, the subject is likely to be infected with the infectious illness contracted by the third party, and
cause the at least one terminal to output a notification regarding the infectious illness estimated such that the subject is likely to be contracted.

18. The health management system according to claim 16, wherein
the second processing circuitry is configured to
estimate that in a case where the illness contracted by the third party is an infectious illness, a person who is a close contact with the third party is included in a first infection preliminary group, and a person who is a close contact with the person included in the first infection preliminary group is included in a second infection preliminary group, and
cause the at least one terminal to output a notification regarding the infectious illness estimated such that the subject is likely to be contracted in a case where the person included in the first infection preliminary group is turned to contract the infectious illness, and the subject is included in the second infection preliminary group.

19. The health management system according to claim 16, wherein
the second processing circuitry is configured to estimate a degree of an infection risk regarding the infectious illness of the subject based on the vital data, the activity history information, and the information on the disease of the third party.

20. The health management system according to claim 19, wherein
the second processing circuitry is configured to increase an acquisition frequency of the vital data and the activity history information as the estimated infection risk increases.
